(19) United States
(12) Patent Application Publication
(10) Pub. No.: US 2010/0100067 A1
(43) Pub. Date: Apr. 22, 2010

(54) Articles with a series of graphical objects providing a functional cue

(76) Inventor: Sebastian C. Pugliese, III, Hortonville, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/288,302

(22) Filed: Oct. 17, 2008

(51) Int. Cl. A61F 13/15

(52) U.S. Cl. 604/378

(57) ABSTRACT

The present disclosure provides an absorbent personal care article having a graphic which creates a visual perception of a functional aspect of the absorbent personal care article. For example, an absorbent personal care article has a personal care absorbent article having an absorbent core and a body-facing surface. A plurality of graphical objects are visible on the surface such that the graphical objects form a pattern illustrating at least one ordered sequence to create a functional cue. This functional cue conveys at least one functional aspect associated with as structural feature of the absorbent personal care article.

### BACKGROUND

1. Field

The present disclosure relates to an article having a series of graphical objects visible from a body-facing surface of the article, the series of graphical objects forming a pattern that provides a functional cue. The functional cue creates an enhanced visual perception of a visible structural feature to help convey a functional aspect of the product to the viewer.

2. General Background

Absorbent personal care articles intended to absorb discharged bodily fluids are well known in the art. Such absorbent articles generally comprise a fibrous mass or other absorbent core which can absorb and hold body fluids. Similarly, it is well known that feminine care articles have been employed to absorb and hold liquids, such as urine, discharge and/or menses. A typical structure of an absorbent article includes a fluid impermeable back sheet, a fluid permeable top sheet and an absorbent core positioned between the back sheet and the top sheet. Prior absorbent articles have also included various other features to improve fluid handling, such as intake layers, distribution layers, retention layers and the like. In these absorbent personal care articles, the top sheet is the body-facing side of the absorbent article and the back sheet is the garment-facing side of the absorbent article. Some articles also often include other structural features which function to provide enhanced product comfort and effectiveness. For example, such products may also include raised or recessed areas, and elastic components, in order to bring portions of the article closer to the consumer's body surface.

The various absorbent personal care article products often provide benefits that are not readily apparent to a user. These benefits may be indiscernibly incorporated internally within the product or may not be easily perceivable or visible on a surface of the product. For example, embossments may be provided to enhance or direct better intake of fluids into the absorbent article, or to provide a folded pad that provides better fit or pad storage for a user. Similarly, apertures may be provided in specific locations to direct fluid transmission within a product or between layers of a product.

Without a visual indication relating to functionality/structure of the products, consumers may select a product offering less functionality than one that would better meet their needs. The consumer may not readily perceive added structural features new to a particular product or technological advancements being utilized. Accordingly, the ability to communicate the improved functionality/structure of the absorbent personal care articles is useful.

There are numerous examples of graphics printed on disposable absorbent articles. Some manufacturers, for example, print various custom characters on diapers. Other manufacturers print the central portion of sanitary pads, or print a race-track like ring around the central portion. In some cases, manufacturers many print flowers or other objects in sanitary articles. However, many of these graphics are meant to solely provide either aesthetic improvements to the absorbent articles, or alternatively, as a limited visual tool to indicate the product's non-visible scent or flora contents.

Consequently, it would be useful for more products to be both visually appealing to the consumer who is purchasing and using them, and also for such visual appeal to be combined with a nonaesthetic role. To appeal to a consumer, it is desirable for a design to be aesthetically pleasing in addition to communicating a physical function/structure. Accordingly, there is a need to provide products that are visually appealing to the consumer while also providing an indication of the product's already visible, but not readily apparent functionality.

### SUMMARY

Generally stated, the present disclosure provides an absorbent personal care article having a graphic which helps convey to an individual viewing the article an enhanced visual perception of a structural feature to help convey a functional aspect of the product to the viewer. It has been found that a series of discrete shapes, such as circles, along a line can serve as a functional cue to a viewer of the series. For example, a substantially monotonic-decreasing series of spaced-apart circles disposed on an absorbent article helps serve as a functional cue of flexibility, directional flow and intake characteristics/structure of the absorbent article. Even though such structure is often visible, but subdued, from the viewing surface of the article or body-facing surface layer of the article, such a cue enhances the message to the consumer of the presence of such structure.

In one embodiment, an absorbent personal care article has a body having an internal absorbent core and a body-facing surface. In the body-facing surface a plurality of graphical objects are visible, wherein the graphical objects form a pattern illustrating at least one ordered sequence to create a functional cue. This functional cue conveys at least one functional aspect associated with use of the absorbent personal care article.

In a further embodiment, the functional cue provides an enhanced visual perception of an already visible structural feature to help convey the functional aspect of the product to the consumer. These structural features can be selected, for example, from embossments, recessed or raised portions, apertures, other such features and combinations thereof.

In an exemplary embodiment, the functional aspect conveyed may be proper directional flow and intake of exudates into the absorbent personal care article and away from the body-facing surface, or better comfort and flexibility during use by a consumer. Such functional aspects may also be the result of raised or lowered portions on the product, or changes in topographical height, which feature is emphasized by the graphical design. Such raised or lowered areas may be the result of a “hump”-like feature on a product that promotes enhanced contact with an individual during use, or alternatively elastic components or fit features, which further enhance the body-contacting capability of the product.

In one embodiment, the functional cue is created by an ordered sequence that is a series of discrete graphical objects. The center of the ordered sequence may be positioned in the central region of the body, that is the regions in the of the absorbent personal care article in the middle of the body-facing layer, wherein subsequent graphical object increase in size along the ordered sequence as the ordered sequence extends out from the central region of the body towards the periphery, that is the regions in the of the absorbent personal care article on the outer edges of the body-facing layer. This configuration may convey a functional cue of a change in height of the body-facing surface in the central region of the body that may include a raised or lowered area in the central region of the absorbent article.

In an exemplary embodiment, subsequent graphical objects in the ordered sequence increase monotonically in size along the ordered sequence as the ordered sequence extends out from the central region of the body towards the periphery of the absorbent personal care article.

In another embodiment, the series of graphical objects visible from the body-facing surface illustrating a plurality of ordered sequences. In this embodiment, the plurality of ordered sequences converges towards a central region of the body to create a functional cue. This functional cue provides a visual perception conveying at least one functional aspect associated with use of the absorbent personal care article. In other embodiments, the plurality of ordered sequences may intersect.

In another embodiment, the series of graphical objects visible from the body-facing surface illustrating a plurality of ordered sequences and may be positioned in the central region of the body, wherein subsequent graphical objects increase in size along the ordered sequence as the ordered sequence extends out from the central region of the body towards the periphery of the absorbent personal care article.

In a further exemplary embodiment, all of the graphical objects have a single common color. In other embodiments, the graphical objects have multiple colors.

In a still further exemplary embodiment, the graphical objects are arranged to enlarge as they approach the center of the article, and decrease in size the further from the center.

In still a further exemplary embodiment, the decrease or increase in size of the graphical objects does not occur in the center of the article, but instead occurs towards a product end regions.

To allow a user to view the functional cue, the graphical objects may be printed onto the body-facing layer. Such printing may occur on the body-facing surface of the body-facing layer or the underlying surface (internally facing surface) of the body-facing layer. In another embodiment, the graphical objects may be printed on a product layer other than the body-surface layer of the product, that is visible through the layer or layers defining the body-facing surface or garment facing surface. For example such graphic design can be printed on a layer of the absorbent component, the backsheet component, or even an adhesive release sheet component that is often found attached to the garment facing layer (backsheet).

In an exemplary embodiment, the graphical objects comprise closed discrete shapes of different sizes that are separated and arranged in a linear or curvilinear arrangement forming an ordered sequence. Exemplary objects may be selected from circles, ovals, triangles, squares, rectangles, flowers, hearts and other such objects, The graphical objects may also be somewhat overlapping in that the graphical objects are not completely contained within one another. In an alternative embodiment, the graphical objects may comprise lines that converge to a single point or towards each other.

In still a further alternative embodiment, the graphical objects are comprised of embossed objects. In still a further alternative embodiment, the graphical objects are comprised of “cut and placed” objects. In still another further embodiment, the graphical objects are comprised of apertured objects. In still another further embodiment, the graphical objects are comprised of a combination of at least two objects selected from printed objects, embossed objects, apertured objects and cut and placed objects.

### BRIEF DESCRIPTION

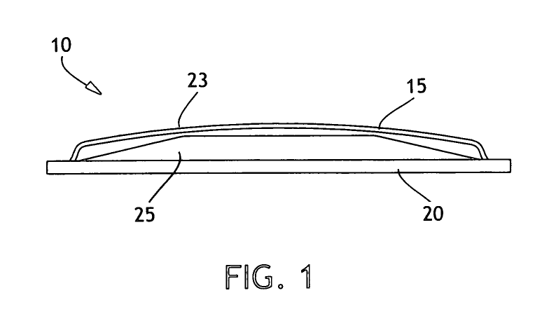

FIG. 1 illustrates a side cut-away view of an embodiment of an absorbent article of the present disclosure.

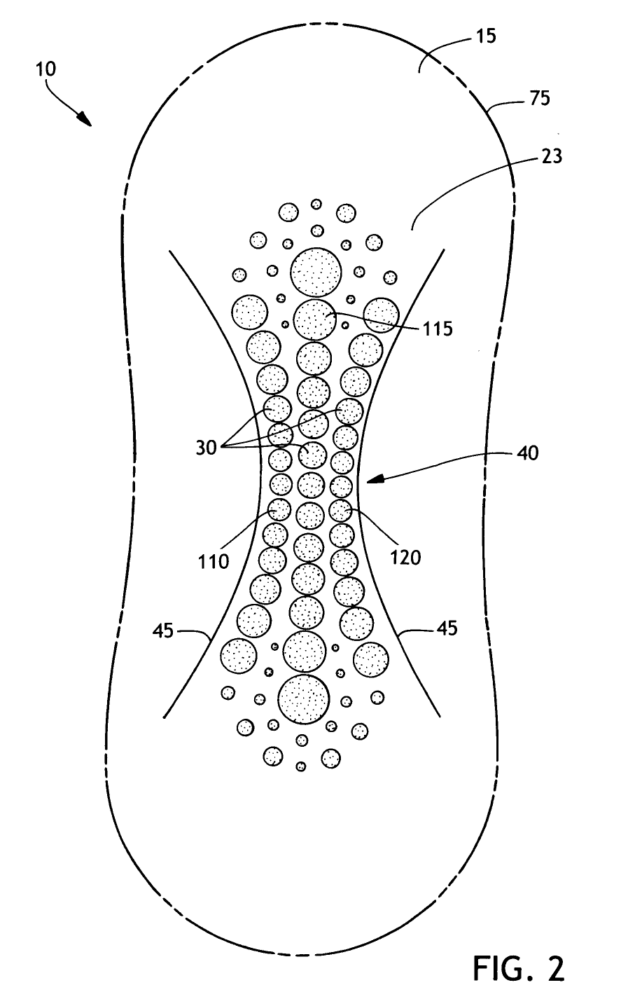

FIG. 2 illustrates a top view of the absorbent article shown of an embodiment of the present disclosure.

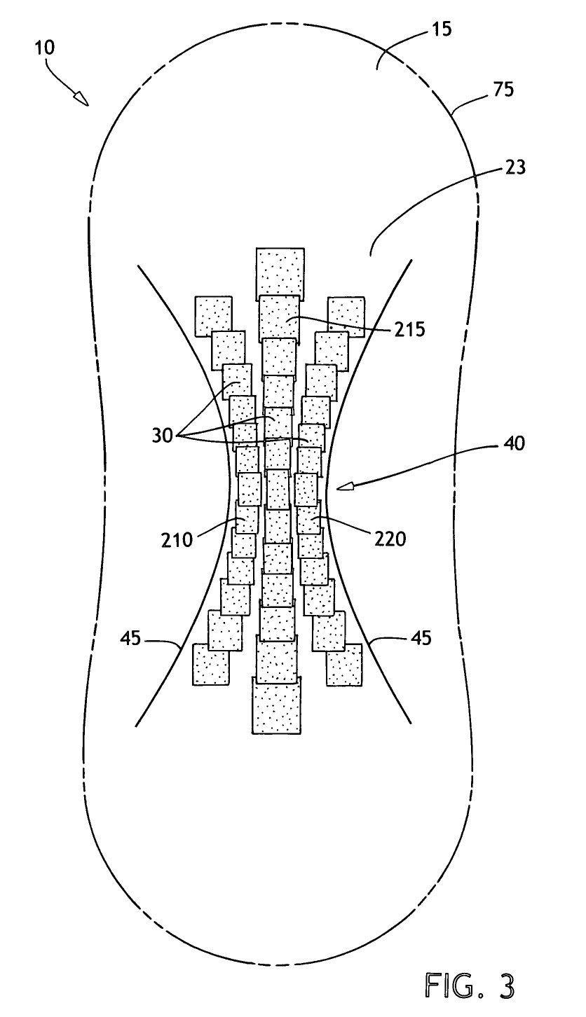

FIG. 3 illustrates a top view of the absorbent article shown of an alternative embodiment of the present disclosure.

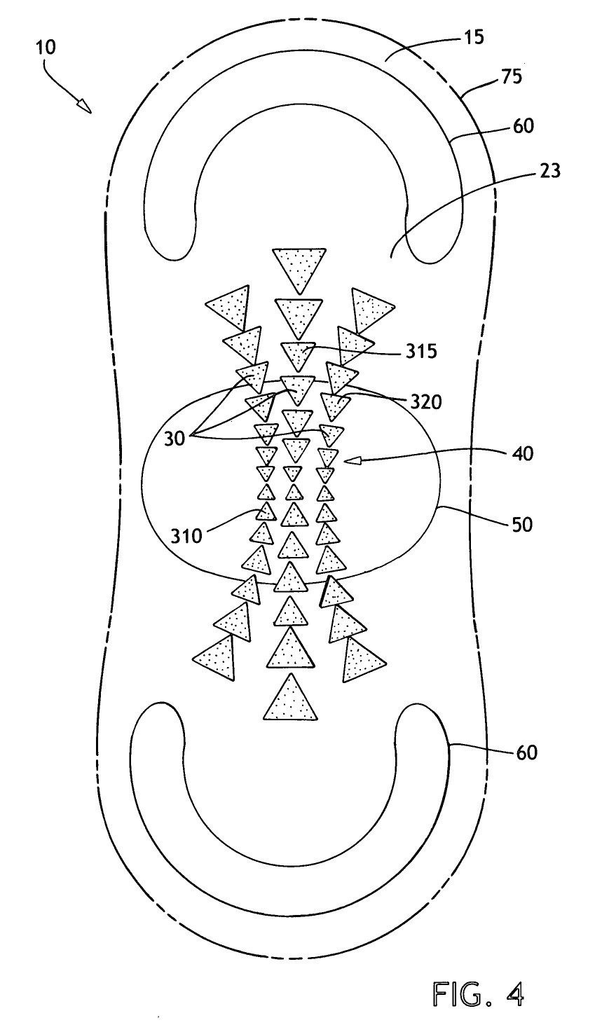

FIG. 4 illustrates a top view of the absorbent article shown of another alternative embodiment of the present disclosure.

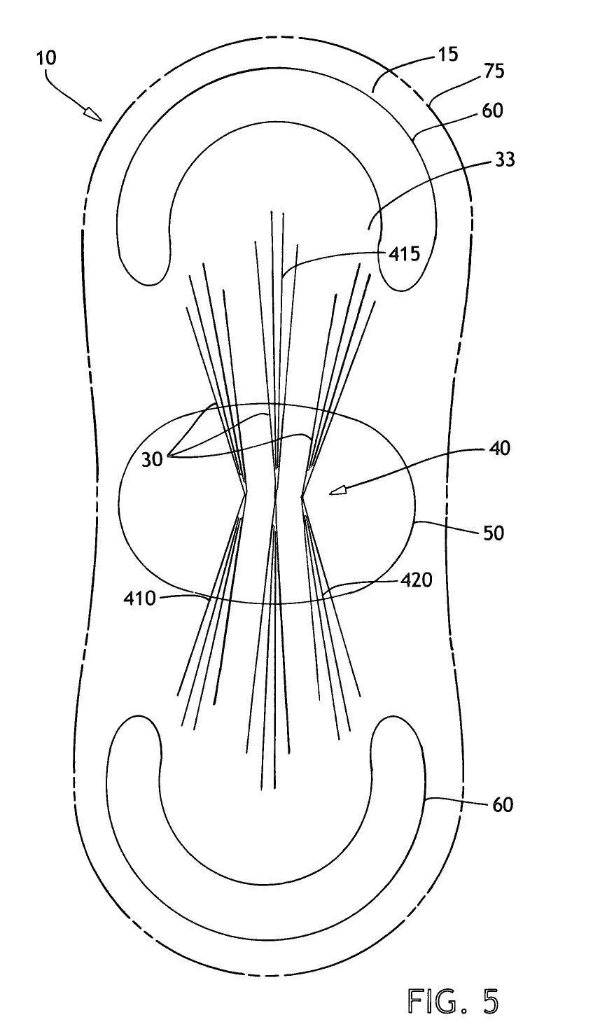

FIG. 5 illustrates a top view of the absorbent article shown of another alternative embodiment of the present disclosure.

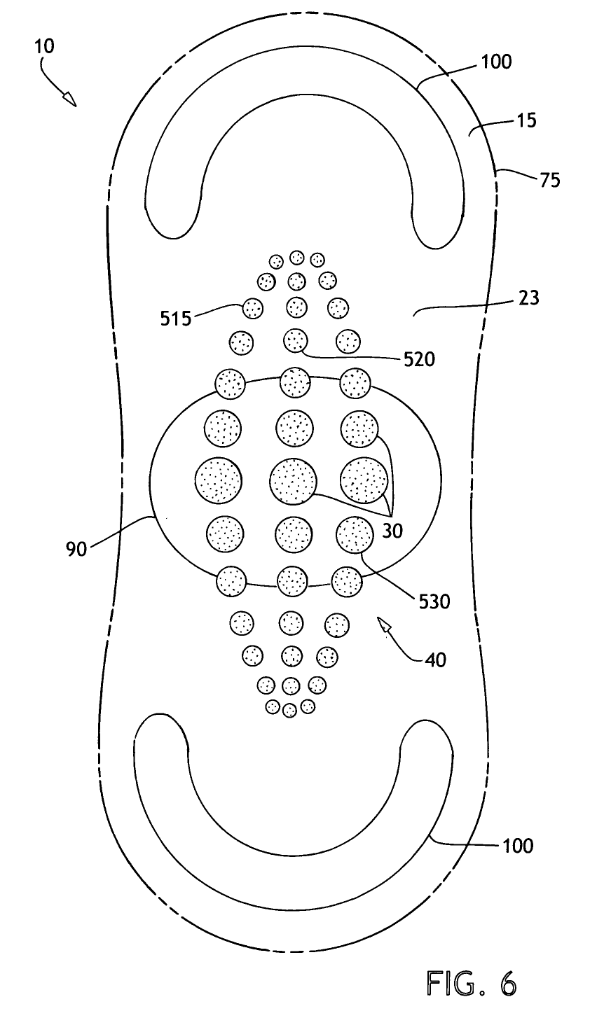

FIG. 6 illustrates the absorbent article shown of another alternative embodiment of the present disclosure.

### DETAILED DESCRIPTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “absorbent product” or “absorbent article”, as used herein, refers to any article used to control bodily fluids that are configured to absorb and retain bodily exudates, including urine, blood, menses, and other bodily discharges, such as sweat and vaginal secretions resulting from sexual activity and the like. In addition, the term is intended to include odor absorbing articles.

As used herein, “body-facing layer” means that layer of the absorbent article which is intended to be disposed toward or placed adjacent to the body of the wearer during ordinary use. The body-facing layer includes a body-facing surface, which is intended to immediately face the body of a consumer while the product is in use. The body-facing layer also includes an internal garment facing surface, being opposite to the body-facing surface. The “garment-facing layer” is on the opposite side of the absorbent article from the body-facing layer. The garment-facing surface includes an outward surface (a garment facing surface) of the absorbent article and is intended to be disposed to face away from the wearer's body during ordinary use. The garment-facing surface is generally arranged to immediately face toward or placed adjacent to the wearer's undergarments when the absorbent article is worn.

As used herein, “monotonically increasing” and “monotonically decreasing” mean that the successive members of a sequence either consistently increase or decrease but do not oscillate in relative value. Each member of a monotonically increasing sequence is greater than or equal to the preceding member; each member of a monotonically decreasing sequence is less than or equal to the preceding member.

As used herein, the term “absorbent core” is intended to mean a configuration of an absorbent material which allows bodily fluids to be absorbed by the absorbent material.

In the following detailed description, reference is made to the accompanying drawings which form a part hereof, and in which is shown by way of illustration, specific aspects in which the disclosure may be practiced. These aspects are described in sufficient detail to enable those skilled in the art to practice the disclosed article, and it is to be understood that other aspects may be utilized and that mechanical, procedural, and other changes may be made without departing from the spirit and scope of the present disclosure. The following detailed description is, therefore, not to be taken in a limiting sense, and the scope of the present disclosure is defined only by the appended claims, along with the full scope of equivalents to which such claims are entitled.

The absorbent personal care product of the present disclosure provides an absorbent article that creates a visual perception that helps convey to a consumer viewing the article the functional capability of the article. Examples of functional aspects that may be shown by this functional cue include directional flow in the absorbent article, and improved fit and comfort for the user of the absorbent article, improved flexibility of the article, or by providing enhanced structure which places the article closer to the user. Structural elements that may be enhanced by such functional cues include, but are not limited to, embossments, recessed or raised areas and apertures on the absorbent personal care article, and combinations thereof.

In an exemplary embodiment, an absorbent personal care article includes a body containing an absorbent core and a body-facing layer. Visible to a user from the body-facing surface is a series of graphical objects wherein the graphical objects form a pattern illustrating at least one ordered sequence. The graphical objects comprise closed discrete shapes of different sizes that are configured in a linear or curvilinear arrangement and if connected at its midpoint would form a line. Exemplary objects may be selected from circles, ovals, triangles, squares, rectangles, flowers, hearts and other such objects. The graphical objects may be separated or also be somewhat overlapping in that the graphical objects are not completely contained within one another. In exemplary embodiments, the at least one ordered sequence conveys at least one functional aspect associated with use of the absorbent personal care article.

To facilitate the functional effect of the at least one functional cue, a series of discrete graphical objects form an ordered sequence having a center-point in the central region of the body. In exemplary embodiment, an ordered sequence is a series of graphical objects or points that are visible on a surface and would form a line if connected at a centralized point whether they are regular or irregular shapes. In other exemplary embodiment, such ordered sequences may not be concentric in their configuration, in that one object does not completely surround another in their configuration, and do not include the same central point. Rather such objects are not centered around one another and are positioned along a linear or curvilinear arrangement.

In an exemplary embodiment, subsequent graphical objects increase in size along the ordered sequence as the ordered sequence extends out from the central region of the body towards the periphery of the absorbent personal care article in at least one direction. The absorbent article is manufactured so that a person viewing the article sees the series of graphical objects along an ordered sequence or functional cue, with the objects substantially increasing in size along the ordered sequence along at least a portion of the sequence. Accordingly, a functional cue is perceived due to the contrast in sizes of the graphical objects. The ordered sequence may include numerous portions of increasing or decreasing sized objects, and may also be positioned in non-central locations on the product. For example, such ordered sequences may help to illustrate raised or lowered areas positioned at the product ends.

Context strongly affects a human's perceptions. In other words, an object's surroundings influence our perception of that object. In a size-relationship or contrast illusion, the proximity of an object to larger objects causes the size of the first object to be underestimated. Size diminution of an object will make the object appear farther away and at a different distance. In one embodiment, the discrete series of objects forming an ordered sequence of the present disclosure decrease in size in one embodiment, as the ordered sequence move towards the central region of the absorbent personal care article. This conveys a perception of a functional cue by virtue of the objects having the same shape of different sizes and simulating a multi-layering, or change in height, of objects in three dimensional space on a two dimensional surface through a size-relationship illusion. Such an ordered sequence may emphasize a central recessed portion (or deeper area) and raised end portions for the consumer. Such features might be visible but not as evident, without such emphasis. Similarly, if the ordered sequence was reversed, larger graphical objects could be positioned in the central region of the product, with smaller graphical objects towards the product end, thereby emphasizing a raised central area surrounded by recessed or lower height areas at a product's ends. In still a further alternative embodiment, the ordered sequence of graphic objects could be presented so as to have either monotonically decreasing or increasing shapes, and also shapes that are illustrated so as to reflect slightly compacted appearances (for example, oval shapes in the case of circular shapes) as they are situated on the elevated or recessed product topography. The monotonically decreasing or increasing sized shapes would have more non-compact shapes in the flatter areas of the product topography, such as at the tops or bottoms of humps or recesses respectively, and the flatter areas of the products.

Accordingly, the functional cue conveyed by the series of objects decreasing in size to a user of the personal care article that proper or increased directional flow of exudates or fluids towards the center of the absorbent personal care article and away from the edges of the absorbent article, and thus the user's body, will occur. Less leakage and performance improvements, the functional aspects of the embossments of the absorbent personal care article for example, may be communicated to a consumer by simply viewing the article.

In an exemplary embodiment, the ordered sequence of graphical objects illustrates a monotonically-increasing sequence as the ordered sequence extends out from the central region of the body towards the periphery of the absorbent personal care article. In a monotonically-increasing sequence, each subsequent graphical object increases in size as the series progresses. The monotonically-increasing sequence creates an ordered size difference between different shapes so that there is an increase in size as the ordered sequence is arranged from the central region of the absorbent personal care article towards the periphery.

In an alternative embodiment, the graphical objects could increase in size as the ordered sequence is arranged from the article periphery to the central region.

In another further embodiment, visible to a user from the body-facing surface, is a series of graphical objects wherein the graphical objects form a pattern illustrating a plurality of ordered sequences creating a functional cue. In exemplary embodiments, the plurality of ordered sequences may converge together towards a single point, converge together without contacting, or converge together to intersect. The convergence of the plurality of ordered sequences may convey at least one functional aspect or visible structural feature associated with use of the absorbent personal care article.

In an exemplary embodiment, to facilitate the functional effect of the functional cue, the plurality of functional ordered sequences formed by discrete graphical objects converge in the central region of the body that conveys a perception of linear perspective. Linear perspective is a phenomenon that in which parallel lines appear to converge as they recede into the distance and appear to meet at a point on the horizon. Lines that converge toward the horizon are an indicator of distance. Therefore, as the plurality of ordered sequences converge into the central region of the personal care absorbent article, a user of the product viewing the personal care absorbent article will see the central region as farther or closer away as the case may be depending on the presence of a recess or hump. In still another further embodiment, such convergence could be illustrated to occur at the product ends rather than in the central region.

Correspondingly, the functional cue conveyed by the plurality of ordered sequences converging in the central region creates a perception to a user of the personal care article that proper or increased directional flow of exudates or fluids towards the center of the absorbent personal care article and into the absorbent core and away from the body-facing surface, and thus the user's body, will occur so as to emphasize the physical presence of embossing patterns.

In addition, the three dimensional perspective conveyed by the plurality of ordered sequences converging in the central region creates a perception to a user of the personal care article that the absorbent personal care article allows a user to move more freely to highlight the article's flexibility and/or better fit within the clothing of the individual to allow for freer movement, such as to emphasize the presence of elastic components which can be seen through layers of the product. For example, the ordered sequences may follow a curvilinear path that more closely approximates the curvature of the inner thigh, and may illustrate the ability of the absorbent article to bend along that curvature or to adhere closely to the body. The perception provided by the graphical elements therefore correlates to activity of consumers to indicate that the absorbent article is designed to enable such movement with comfort and ease.

In another embodiment, a combination of the multiple ordered sequences converging in the central region wherein each ordered sequence includes a plurality of graphical objects, wherein subsequent graphical objects increase in size along the ordered sequence as the ordered sequence extends out from the central region of the body towards the periphery of the absorbent personal care article. Therefore, a functional cue is created by both a size-relationship illusion and a linear perspective created by the plurality of ordered sequences. By combining the two functional cues, the user is better able to correlate improvements in visible structural technology, and function that may not be emphasized by just viewing the structure.

To gain a better understanding of the present disclosure, attention is directed to the figures of the present specification. FIG. 1 shows a cross-section of an exemplary absorbent personal care article 10. In the illustrated embodiment, the personal care article 10 includes a liquid permeable top sheet layer 15 or cover layer, a substantially liquid impermeable back sheet layer 20 joined to the top sheet layer 15, and an internal absorbent core 25 positioned and held between the body-facing layer 15 and the back sheet layer 20. As shown, the liquid permeable body-facing layer 15 and the liquid impermeable back sheet layer 20 provide exemplary exterior surfaces of the absorbent personal care article 10. The absorbent personal care article 10 may also include other components, such as liquid wicking layers, liquid intake layers, liquid distribution layers, transfer layers, and barrier layers, as well as combinations thereof. In the exemplary embodiment, the body-facing layer 15 and back sheet 20 provides the absorbent personal care article 10 respectively with a body-facing surface 23 and a garment-facing surface.

In some embodiments, the body-facing layer 15 includes a woven fabric, a nonwoven fabric, a polymer film, a film-fabric laminate, as well as combinations thereof. Examples of a nonwoven fabric include spunbond fabric, meltblown fabric, coform fabric, a carded web, a bonded-carded-web, a bicomponent spunbond fabric as well as combinations thereof. A more particular example of a suitable material for the body-facing layer 15 includes a bonded-carded-web composed of polypropylene and polyethylene. Other examples of suitable materials include composite materials of a polymer and a nonwoven fabric material. The composite materials may be in the form of integral sheets formed by the extrusion of a polymer onto a web of spunbond material. A plurality of pores, perforations, apertures or other openings, as well as combinations thereof, which are present or formed in the body-facing layer 15, may provide the liquid-permeability. The body-facing layer 15 may retain little or no liquid in its structure and be configured to provide a comfortable and non-irritating surface next to the body of the user.

For some embodiments, the back sheet 20 includes a polymeric film, a woven fabric, a nonwoven fabric, as well as combinations or composites thereof. For example, the back sheet 20 may include a polymer film laminated to a woven or nonwoven fabric. The polymer film may be composed of polyethylene, polypropylene, polyester, as well as combinations thereof. The back sheet 20 may permit passage of air and moisture vapor out of the absorbent personal care article 10 while blocking the passage of bodily liquids. An example of a suitable material includes a breathable, microporous film. Bicomponent films or other multi-component films may also be used, as well as woven and/or nonwoven fabrics which have been treated to render them liquid-impermeable. Another suitable material for the back sheet 20 includes closed cell foam materials.

Structure of the internal absorbent core 25 provides desired levels of liquid retention and storage capacity and other non-illustrated layers may provide desired levels of liquid acquisition and distribution. The internal absorbent core 25 holds a liquid, such as urine, menses, other complex biological liquid, as well as combinations thereof. The internal absorbent core 25 may include a matrix of absorbent fibers and/or absorbent particulate material, and the absorbent fiber may include natural and/or synthetic fiber. The internal absorbent core 25 may also include one or more components that modify menses or inter-menstrual liquids. The internal absorbent core 25 may also include superabsorbent material, such as a water-swellable, generally water-insoluble, hydrogel-forming polymeric absorbent material, which is capable of absorbing at least about 20, about 30, or about 60 times or more its weight in physiological saline (e.g., 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, and polyvinyl pyridine. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may occur by irradiation or covalent, ionic, Van der Waals, or hydrogen bonding.

In one embodiment, printing an ink onto the body-facing layer, for example the body-facing surface 23 of the body-facing layer 15 or garment facing surface of the body-facing layer would provide the visual graphic image. Alternatively the printing may occur on a layer visible under the body-facing layer 15 (i.e., the body-facing layer 15 may be transparent or translucent). This provides one method of integrating the graphical objects into the absorbent personal care article 10 or any other substrate. The graphical objects may thus be patterned on an internal part of the absorbent personal care article 10 such that they are protected from rubbing off while still being visible through to the exterior surface. In further embodiments, the graphical objects may be printed on the backsheet or layers over the backsheet, such as adhesive release sheets. For some embodiments, an ink jet printer applies the graphical objects. Other acceptable printing techniques include flexographic and graveur roll printing. While directly printed graphics are economical and time efficient, embossing and/or slitting/aperturing techniques also enable making the graphical objects visible. A combination of these techniques is also contemplated. Further such graphical objects may be introduced into the product structure by cutting and placing pre-formed (off-line) layers including such designs, or individual shapes into the product during product manufacture.

With reference to FIG. 2, the body-facing surface 23 of the body-facing layer 15 of an exemplary absorbent personal care article 10 is illustrated. In this embodiment, a plurality of discrete graphical objects 30 is printed on the body-facing surface 23 of the absorbent personal care article 10. In this exemplary embodiment, the discrete graphical objects 30 are circles or bubbles printed onto the body-facing surface of the body of the personal care absorbent article 10. In this particular embodiment, there exists three series of graphical objects, each series illustrating an ordered sequence 110, 115, 120 to help convey a functional cue of the flexibility and decreased leakage of the article, shown in conjunction with visibly seen embossing channels 45.

In this particular embodiment, the functional cue created by the plurality of ordered sequences 110, 115, 120 enhances the perception of the structural feature of flexibility and decreased leakage caused by embossing channels 45 in the absorbent article 10. The embossing channels 45 which are channels adjacent to the longitudinal edges are formed by compressing the layers of the absorbent article 10 a sufficient amount to remain intact throughout the intended use of the absorbent article 10. It is relevant to note that these embossed channels 45 are not barrier lines, that is, lines which are intended to stop the flow of body fluids beyond their boundary. Rather, the embossing lines 45 are designed to reduce leakage by pooling body fluids during use. These lines may be produced in any manner such as by the application of heat, including hot calendar embossing or by ultrasonic means.

The embossing lines 45 along each longitudinal side of the absorbent article 10 may be a straight line running parallel to the length of the absorbent article 10 and extending the length of the absorbent article 10 or, preferably, the embossing lines 45 may be arcuate wherein the midpoint of the arc of the embossed channel is the greatest distance from the longitudinal edge of the absorbent article 10 as demonstrated in FIG. 2. The embossing lines 45 may extend almost the complete longitudinal length of the pad.

The arcuate embossed channels are spaced inwardly of the longitudinal axis of the body-facing surface 23 of the absorbent article 10. That is, the area near the center of the arcuate embossed channels is the greatest distance from the longitudinal edge of the absorbent article 10.

When the absorbent article is worn, overflow from the central product will advantageously be absorbed by lateral flaps or wings (not shown) protecting the clothing against being soiled and giving the wearer increased confidence in avoiding embarrassing occurrences. The absorbent article 10 itself may be a uniform width. Alternatively, the absorbent article could have an hourglass shape or other shape and the same advantages could thereby be obtained. In the case of the hourglass shaped absorbent article, the width of the absorbent article 10 is less in the central region.

In each of the ordered sequences 110, 115, 120, the graphical objects 30 increase in size monotonically as the ordered sequence is arranged from the central region 40 of the body towards the periphery 75 of the absorbent personal care article 10. In addition, each of the ordered sequences 110, 115, 120 converge towards the central region 40 of the personal care absorbent article 10. Thus, the ordered sequences convey proper or increased directional flow of exudates or fluids towards the center of the absorbent personal care article and away from the periphery 75 of the absorbent article 10 enhancing the perception of the functionality of the embossing lines 45 to a consumer.

The patterns created by the ordered sequences 110, 115, 120 are superior in that not only do they provide functional cues of flexibility and decreased amount of leaking, but the graphical objects 30 also provide a functional cue of increased intake by the absorbent pad, based on the visible embossing lines.

In the embodiments shown, the graphical objects 30 are shaded with a single color tone to provide the pattern. It should be recognized that multiple color tones may be utilized as well.

The ordered sequences created by the series of graphical objects can be in any configuration that portrays a functional cue. For example, the ordered sequence can be in straight, curved, sinusoidal, or any other configuration of lines. As shown by the exemplary embodiment in FIG. 2 where the central ordered sequence 115 is straight, and the outer ordered sequences 110, 120 are curved; a combination of different configurations may be utilized to optimize the functional cue of the product's ability to bend and provide comfort and or intake.

The graphical objects 30 may be printed discretely anywhere onto the body-facing layer 15 of the absorbent personal care article 10, and are not typically bounded by an outline or area for printing. Many previous absorbent care articles have included graphical illustrations that are bounded by an outer line or area. By allowing the graphical objects 30 to be printed without a bounded area, the discrete objects provide a manufacturing benefit since allowable variation for print patterns of discrete objects on an individual article may be greater than larger print images having a continuous border. That is, it may be true that a viewer, looking at, for example, a large printed design at the center of a pad may more readily detect when the design is offset from the center, compared to a series of smaller, discrete objects printed along a ordered sequence printed elsewhere on the surface of the article.

FIG. 3 illustrates the body-facing layer 15 of an alternative embodiment of a personal care absorbent article. In this exemplary embodiment, the discrete graphical objects 30 are squares printed onto the body-facing surface 23 of the personal care absorbent article 10 of the personal care absorbent article 10. In this particular embodiment, there exists three series of graphical objects, each series illustrating an ordered sequence 210, 215, 220 to help convey a functional cue.

As illustrated by this embodiment, the graphical objects 30 may be of different shapes to provide both the functional cue, but also are designed to be aesthetically pleasing. The graphical objects 30 may be any form, open or closed shape, or line, that provides both of these benefits.

Additionally, the graphical objects 30 may or may not overlap. As shown by FIG. 3, each ordered sequence 210, 215, 220 is defined by graphical objects 30 that overlap along the ordered sequence 210, 215, 220. In other embodiments, each graphical object is a separate shape that is not touching or overlapping any other graphical object. For example, such sequence may comprise a series of holiday or ornamental designs, such as progressively enlarged floral shapes, heart shapes or shamrocks.

The functional cue created by the plurality of ordered sequences 210, 215, 220 enhances the perception of the structural feature of flexibility and decreased leakage caused by embossing lines 45 in the absorbent article 10. Thus, similar to FIG. 2, the ordered sequences convey proper or increased directional flow of exudates or fluids towards the center of the absorbent personal care article and away from the periphery 75 of the absorbent article 10 enhancing the perception of the functionality of the embossing lines 45 to a consumer.

FIG. 4 illustrates the body-facing surface of another alternative embodiment of a personal care absorbent article. In this exemplary embodiment, the discrete graphical objects 30 are triangles printed onto either side the body-facing surface of the personal care absorbent article 10. In this particular embodiment, there exists three series of graphical objects, each series illustrating an ordered sequence 310, 315, 320 to help convey a functional cue.

In this particular embodiment, the functional cue created by the plurality of ordered sequences 310, 315, 320 enhances the perception of the structural feature of better directional flow of fluids caused by a recessed central portion 50 in the absorbent article 10. The absorbent article 10 is formed to have a recessed central portion from the outer edges of the absorbent article 10. The central recess portion 50 is designed to reduce leakage by pooling body fluids during use towards the center of the absorbent article. This may be enhanced by forming raised portions 60 on the outer edges of the absorbent article 10 to further promote flow of fluids to the center of the absorbent article 10.

The combination of different sized graphical objects 30 and convergence of the plurality of ordered sequences 310, 315, 320 may create a functional cue of a change in height in the central region of the personal care absorbent article 10. Thus, the plurality of ordered sequences 310, 315, 320 conveys directional flow of exudates or fluids towards the center of the absorbent personal care article and away from the periphery 75 of the absorbent article to enhance awareness of the functionality of the visible recessed central portion 50 of the absorbent article 10.

This embodiment illustrates that the graphical objects 30 may be oriented in the same direction or in different directions. In fact, orientation of the graphical objects 30 may enhance the implication provided by the functional cue. In FIG. 4, all of the graphical objects 30 are triangle shapes pointing towards the central region of the absorbent article 10. This orientation indicates to a user viewing the absorbent article directional flow of fluids will go towards the center of the absorbent article in addition to the functional cue provided by a monotonic-decrease in the size of each graphical object in each ordered sequence towards the center and a plurality of ordered sequences converging towards the center.

FIG. 5 illustrates the body-facing surface 23 of another alternative embodiment of a personal care absorbent article. In this exemplary embodiment, the discrete graphical objects 30 are lines printed on to the body-facing surface 23 of the personal care absorbent article 10. In this particular embodiment, there exists three series of graphical objects, each series illustrating an ordered sequence 410, 415, 420 to help convey a functional cue.

In this embodiment, each ordered sequence 410, 415, 420 is a plurality of lines converging to a single point. Similar to the monotonic-decrease is size towards the center in the other illustrated embodiment, a user of the product viewing the personal care absorbent article will see the central region as farther away as the lines converge to a single point near the central region of the absorbent article. Thus, the plurality of ordered sequences 410, 415, 420 conveys directional flow of exudates or fluids towards the center of the absorbent personal care article and away from the periphery 75 of the absorbent article to enhance awareness of functionality of the visible recessed central portion 50 of the absorbent article 10.

FIG. 6 illustrates the body-facing surface 23 of another alternative embodiment of a personal care absorbent article. In this exemplary embodiment, the discrete graphical objects 30 are lines printed on to the body-facing surface 23 of the personal care absorbent article 10. In this particular embodiment, there exists three series of graphical objects, each series illustrating an ordered sequence 515, 520, 530 to help convey a functional cue of a raised central portion or a thicker central portion.

In each of the ordered sequences 515, 520, 530, the graphical objects 30 decreased in size monotonically as the ordered sequence is arranged from the central region 40 of the body towards the periphery 75 of the absorbent personal care article 10. In addition, each of the ordered sequences 515, 520, 530 converge towards the outer edges of the personal care absorbent article 10. In this embodiment, a thicker or raised portion 90 provides with superior absorbency and a high resistance to leakage due to being placed closer to the body. The functional cue conveyed by these ordered sequences enhances the observation of the existence of a thicker portion or a raised portion 90 in the central region 40 of the absorbent article 10.

It should be understood that the various embodiments described herein are merely illustrative. Having described the disclosure in detail, it will be apparent that modifications and variations are possible without departing from the scope of the disclosure defined in the appended claims.

1. An absorbent personal care article, comprising:
a body having an absorbent core and a body-facing layer; and
a plurality of graphical objects visible from the body-facing layer, wherein the graphical objects form a pattern illustrating at least one ordered sequence to create a functional cue, the functional cue conveying at least one functional aspect of a structural element associated with use of the absorbent personal care article.

2. The absorbent personal care article of claim 1 wherein the structural element is selected from embossments, recessed or raised areas, or apertures on the absorbent personal care article, and combinations thereof.

3. The absorbent personal care article of claim 1 wherein the functional cue conveys directional flow of fluids towards a central portion of the body-facing layer.

4. The absorbent personal care article of claim 1 wherein the functional cue conveys better comfort and flexibility during use by a consumer.

5. The absorbent personal care article of claim 1 wherein the functional cue conveys topographical height in the absorbent personal care article.

6. The absorbent personal care article of claim 1 wherein the ordered sequence is a series of discrete graphical objects, the center of the ordered sequence positioned in a central region of the body wherein subsequent graphical object increase in size along the ordered sequence as the ordered sequence extends out from the central region of the body towards the periphery of the absorbent personal care article.

7. The absorbent personal care article of claim 6 wherein the size increase of the graphical objects provides a functional cue.

8. The absorbent personal care article of claim 6 wherein subsequent graphical objects increase monotonically in size along the ordered sequence as the ordered sequence extends out from the central region of the body towards the periphery of the absorbent personal care article.

9. The absorbent personal care article of claim 1 wherein the graphical objects form a pattern illustrating a plurality of ordered sequences.

10. The absorbent personal care article of claim 9 wherein the plurality of ordered sequences converge in the central region of the body to convey a functional cue.

11. The absorbent personal care article of claim 1, wherein all the graphical objects have a single common color tone.

12. The absorbent personal care article of claim 1, wherein the graphical objects are printed onto the body-facing layer.

13. The absorbent personal care article of claim 1, wherein the graphical elements are patterned on an internal part of the absorbent personal care article visible through a layer defining the body-facing surface.

14. The absorbent personal care article of claim 1, wherein the graphical objects comprise closed discrete shapes of different sizes.

15. The article of claim 1, wherein the graphical objects comprise lines converging towards a single point.

16. The article of claim 1 wherein the graphical objects are overlapping.

17. An absorbent personal care article, comprising:
a body having an internal absorbent core and a body-facing surface; and
a series of graphical objects visible from the body-facing surface, wherein the graphical objects form a pattern illustrating a plurality of ordered sequences, the plurality of ordered sequences converging towards a central region of the personal care absorbent article to create a functional cue, the functional cue conveying at least one structural element associated with use of the absorbent personal care article.

18. The absorbent personal care article of claim 17 wherein the ordered sequence is a series of discrete graphical objects, the center of the ordered sequence positioned in the central region of the personal care absorbent article, wherein subsequent graphical object increase in size along the ordered sequence as the ordered sequence extends out from the central region of the absorbent personal care article towards the periphery of the absorbent personal care article.

19. The absorbent personal care article of claim 17 wherein the series of discrete graphical objects are arranged to convey a topographical height in the absorbent personal care article.

20. The absorbent personal care article of claim 17 wherein subsequent graphical object increase monotonically in size along the ordered sequence as the ordered sequence extends out from the central region of the personal care absorbent article towards the periphery of the absorbent personal care article.

21. The absorbent personal care article of claim 17 wherein all the graphical objects have a single common color tone.

22. The article of claim 17 wherein the graphical objects are printed onto the body-facing layer.

23. The article of claim 17 wherein the graphical elements are patterned on an internal part of the personal care absorbent article visible through a layer defining the body-facing surface.

24. The article of claim 17 wherein the graphical objects comprise closed discrete shapes of different sizes.
